(19) United States
(12) Patent Application Publication
(10) Pub. No.: US 2021/0046025 A1
(43) Pub. Date: Feb. 18, 2021

(54) Injection for Protecting Ischemic Myocardium and Preparation Method thereof

(71) Applicants: Yida TANG, Beijing (CN); Na CHU, Beijing (CN); Yan QIN, Beijing (CN)

(72) Inventors: Yida TANG, Beijing (CN); Na CHU, Beijing (CN); Yan QIN, Beijing (CN)

(21) Appl. No.: 16/964,995

(22) PCT Filed: Jan. 22, 2019
(86) PCT No.: PCT/CN2019/072710

§ 371 Date: Jul. 26, 2020

(30) Foreign Application Priority Data
Jan. 26, 2018 (CN) 201810076352.3

(51) Int. Cl. A61K 31/167; A61K 47/12; A61K 47/10; A61K 47/24; A61K 47/44; A61K 9/00; A61P 9/10

(52) U.S. Cl. CPC A61K 31/167; A61K 47/12; A61K 47/10; A61P 9/10; A61K 47/44; A61K 9/0019; A61K 47/24

(57) ABSTRACT

The present invention relates to an injection for protecting ischemic myocardium, and preparation method for the injection. The emulsion for injection comprises, in parts by weight: 1-5 parts of N-suberoylanilide hydroxamic acid, 0.2-12.5 parts of n emulsifying agent, 2-100 parts of oil for injection, 0.02-5 parts of a solubilizer, 0.03-0.4 part of oleic acid, 0.4-12.5 parts of glycerinum, and the balance being water for injection. The emulsion for injection can effectively be used for increasing local drug concentration of the N-suberoylanilide hydroxamic acid in lipophilic organs such as angiocarpy and/or tissues before a cardiac interventional operation, increasing the bioavailability of the N-suberoylanilide hydroxamic acid, reducing general side effects of the N-suberoylanilide hydroxamic acid, realizing effective protection on myocardium under an ischemic or reperfusion injury state, and reducing or avoiding the occurrence of myocardial infarction. Moreover, the emulsion for injection also provides a novel optional dosage form for application of the N-suberoylanilide hydroxamic acid in treatment of cancers.

### TECHNICAL FIELD

The present invention belongs to the field of pharmaceutical preparations, and relates to an injection for protecting ischemic myocardium and a preparation method thereof; in particular, the present invention relates to an injection emulsion containing N-suberoylanilide hydroxamic acid (SAHA) for protecting ischemic myocardium and a preparation method thereof.

### BACKGROUND

Histone deacetylase inhibitors (HDACI) are a class of compounds that have the function of interfering with the histone deacetylases. There have been a lot of research reports on its treatment of HIV, malignant tumors or chronic fibrotic diseases.

N-suberoylanilide hydroxamic acid (SAHA, which chemical structure is shown in the following formula I) is known as a histone deacetylase (HDAC) inhibitor, which can play a therapeutic role by inducing cell differentiation, blocking cell cycle and inducing cell regulation. In vitro studies have shown that N-suberoylanilide hydroxamic acid can inhibit activities of HDAC 1, HDAC 2, HDAC 3 (type I) and HDAC 6 (type II) enzymes at nanomolar concentrations (IC₅₀<86 nmol/L). In some cancer cells, normal cells can be activated by inhibiting excessive HDAC enzymes. Therefore, N-suberoylanilide hydroxamic acid is helpful for alleviating or terminating the activation of growth genes of certain cancer cells by reducing the activity of HDAC.

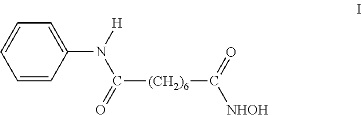

At present, N-suberoylanilide hydroxamic acid has been approved for marketing by the US Food and Drug Administration (FDA) under the trade name Vorinostat, and is used to treat cutaneous T-cell lymphoma (CTCL, a type of T-cell cancer that affects the type of white blood cells in the skin) that are exacerbated, persistent and relapsed, or ineffective after a treatment by two systemic drugs.

Recent studies have also shown that N-suberoylanilide hydroxamic acid can be delivered to reduce myocardial infarction area in large animal models in case of clinically relevant reperfusion. During ischemia/reperfusion, N-suberoylanilide hydroxamic acid produces cardioprotection at least partially by inducing autophagic flux (Min Xie et al., Histone Deacetylase Inhibition Blunts Ischemia/Reperfusion Injury by Inducing Cardiomyocyte Autophagy, Circulation. 2014; 129: 1139-1151). Therefore, N-suberoylanilide hydroxamic acid has important clinical application value for myocardial protection in case of ischemia or reperfusion injury, such as reducing the occurrence of myocardial infarction during cardiac interventional surgery.

However, currently commercially available N-suberoylanilide hydroxamic acid preparations are limited to oral antitumor dosage forms. Due to the poor water solubility of N-suberoylanilide hydroxamic acid and the obvious first-pass metabolism elimination of oral N-suberoylanilide hydroxamic acid, its oral bioavailability is very low and it cannot form an effective therapeutic concentration in lipophilic tissues such as cardiovascular tissue. In addition, N-suberoylanilide hydroxamic acid can bring about a therapeutic effect only when used as a prototype, but its metabolites are inactive, so its oral bioavailability cannot be improved by chemical derivatization. In addition, N-suberoylanilide hydroxamic acid may also cause systemic side effects if taken before cardiac intervention operation.

The above problems in the prior art greatly limit the clinical application of N-suberoylanilide hydroxamic acid, especially its application to myocardial protection during ischemia/reperfusion, since it cannot form an effective local drug concentration in cardiac tissue before cardiac intervention operation. Therefore, there is an urgent need to develop a new pharmaceutical dosage form of N-suberoylanilide hydroxamic acid in order to solve the problem that the local drug concentration of N-suberoylanilide hydroxamic acid in cardiac tissue is too low during the protection of ischemic myocardium.

### SUMMARY

In order to overcome the above deficiencies in the prior art, an object of the present invention is to provide an injection emulsion for protecting ischemic myocardium and a preparation method thereof. The injection emulsion can effectively be used to increase local drug concentration of N-suberoylanilide hydroxamic acid in lipophilic organs and/or tissues such as cardiovascular organs and/or tissue before cardiac intervention operation, increase the bioavailability of N-suberoylanilide hydroxamic acid, reduce general side effects of N-suberoylanilide hydroxamic acid, achieve effective protection on myocardium under an ischemia or reperfusion injury state, and reduce or avoid the occurrence of myocardial infarction. Moreover, the injection emulsion also provides a novel optional dosage form for the application of N-suberoylanilide hydroxamic acid in treatments of cancer and the like.

Technical solutions for achieving the above object are as follows:

An injection emulsion for protecting ischemic myocardium, in parts by weight, containing:

1˜5 parts of N-suberoylanilide hydroxamic acid;

0.2˜12.5 parts of emulsifier;

2˜100 parts of injection oil;

0.02˜5 parts of solubilizer;

0.03˜0.4 parts of oleic acid;

0.4˜12.5 parts of glycerol; and

balance: injection water.

Preferably, in parts by weight, the injection emulsion contains:

3 parts of N-suberoylanilide hydroxamic acid;

0.5˜5 parts of emulsifier;

5˜40 parts of injection oil;

0.05˜2 parts of solubilizer;

0.03˜0.3 parts of oleic acid;

1.0˜5 parts of glycerin; and

balance: injection water.

More preferably, in parts by weight, the injection emulsion contains:

3 parts of N-suberoylanilide hydroxamic acid;

1.0˜2.5 parts of emulsifier;

10˜20 parts of injection oil;

0.1˜1 parts of solubilizer;

0.05˜0.15 parts of oleic acid;

2.0˜2.5 parts of glycerin; and

balance: injection water.

Further preferably, in parts by weight, the injection emulsion contains:

3 parts of N-suberoylanilide hydroxamic acid;

1.0˜2.5 parts of emulsifier;

10˜20 parts of injection oil;

0.6 parts of solubilizer;

0.05˜0.15 parts of oleic acid;

2.0˜2.5 parts of glycerin; and

balance: injection water.

Still further preferably, in parts by weight, the injection emulsion contains:

3 parts of N-suberoylanilide hydroxamic acid;

2 parts of emulsifier;

20 parts of injection oil;

0.6 parts of solubilizer;

0.1 parts of oleic acid;

2.5 parts of glycerin; and

balance: injection water.

In the above injection emulsion, the emulsifier is preferably a phospholipid; more preferably, the phospholipid is selected from one or more of soybean phospholipid, lecithin, hydrogenated soybean phospholipid, or hydrogenated lecithin; further preferably, the phospholipid is soybean phospholipid and/or lecithin.

In the above injection emulsion, the injection oil may be selected from one or more of injection soybean oil, injection safflower oil, injection cottonseed oil, injection sesame oil, injection tea oil, injection olive oil, or injection medium-chain oil; preferably, the injection oil is injection soybean oil.

In the above injection emulsion, the solubilizer may be selected from one or more of Tween-80, propylene glycol, poloxamer 188, or polyethylene glycol 15 hydroxystearate (Solutol HS 15); preferably, the solubilizer is Tween-80 and/or propylene glycol.

According to a specific embodiment of the present invention, in parts by weight, the injection emulsion contains:

3 parts of N-suberoylanilide hydroxamic acid;

2 parts of soybean phospholipid or lecithin;

20 parts of injection soybean oil;

0.6 parts of Tween-80 or propylene glycol;

0.1 parts of oleic acid;

2.5 parts of glycerin; and

balance: injection water.

The invention also provides a preparation method of the injection emulsion, which includes the following steps:

(1) adding a solubilizer and glycerol to N-suberoylanilide hydroxamic acid, heating, and then adding injection water to dissolve N-suberoylanilide hydroxamic acid;

(2) adding an emulsifier to the solution obtained in step (1) and uniformly mixing to obtain an aqueous phase;

(3) uniformly mixing oleic acid with an injection oil to obtain an oil phase;

(4) shear-mixing the aqueous phase and the oil phase at a high speed while heating to obtain a raw emulsion; and

(5) homogenizing the raw emulsion under pressure to obtain the injection emulsion.

In step (1) of the preparation method, preferably, the heating is performed until reaching 50° C. to 90° C., more preferably 70° C. to 80° C.

In step (2) of the preparation method, preferably, the uniformly mixing is performed by shear-mixing; preferably, the speed of the shear-mixing is 3000-10,000 rpm; more preferably, the speed of the shear-mixing is 5000-6000 rpm.

In step (4) of the preparation method, preferably, the heating is performed until reaching 70° C. to 80° C. and the shearing speed of the high-speed shear-mixing is 3000-10,000 rpm, preferably 5000-6000 rpm; preferably, the high-speed shear-mixing is performed for 10 to 40 minutes, more preferably for 25 to 40 minutes, and most preferably for 30 minutes.

In step (5) of the preparation method, preferably, the pressure is 400-4200 bar, more preferably 700-900 bar; and the homogenizing may be performed for 1 to 6 times, preferably 5 to 6 times.

Preferably, the preparation method further includes the following steps:

(6) encapsulating; and

(7) sterilizing.

Preferably, the sterilizing is performed for 30 minutes at 115° C.

In another aspect, the present invention provides an application of the injection emulsion in preparing a medicine for protecting ischemic myocardium, a medicine for treating myocardial ischemia/reperfusion injury, or a medicine for treating myocardial infarction.

In yet another aspect, the present invention provides an application of N-suberoylanilide aniline hydroxamic acid emulsion in preparing a medicine for treating ischemia/reperfusion injury. The present invention can also play a therapeutic role in case of the ischemia/reperfusion injury of cerebral blood vessels and the ischemia/reperfusion injury of other human blood circulation systems.

In yet another aspect, the present invention provides an application of N-suberoylanilide hydroxamic acid emulsion in preparing a medicine for treating cardiovascular diseases.

In yet another aspect, the present invention provides an application of N-suberoylanilide hydroxamic acid emulsion in preparing a medicine for treating heart diseases.

In yet another aspect, the present invention provides an application of N-suberoylanilide hydroxamic acid emulsion in preparing a medicine for protecting ischemic myocardium or a medicine for treating myocardial ischemia/reperfusion injury or a medicine for treating myocardial infarction.

In yet another aspect, the present invention provides an application of N-suberoylanilide hydroxamic acid injection emulsion in preparing a medicine for protecting ischemic myocardium or a medicine for treating myocardial ischemia/reperfusion injury or a medicine for treating myocardial infarction.

Through extensive research, the inventors have found that the injection emulsion can be used as an effective means to effectively improve the bioavailability of N-suberoylanilide hydroxamic acid in lipophilic organs and/or tissues such as cardiovascular organs and/or tissue. However, N-suberoylanilide hydroxamic acid (Vorinostat) is a drug which is insoluble in both oil and water. During the experiment, the inventor found that the emulsion prepared by conventional injection prescriptions has poor stability, obvious drug precipitation occurs after the homogenizing is completed, and an obvious drug powder layer can also be seen after high-speed centrifugation. Therefore, it is necessary to consider adding a solubilizer and a stabilizer to improve the stability of the injection emulsion, thereby ensuring the effect of improving an effective concentration and bioavailability of N-suberoylanilide hydroxamic acid in cardiac tissue.

Importantly, the inventors have found that for this particular poorly-soluble drug, not all solubilizers and/or stabilizers can improve the stability of the obtained injection emulsion, while in the presence of the solubilizer and stabilizer selected by the present invention, an injection emulsion with the best stability can be obtained. In addition, the inventors also noticed that the stability of the injection emulsion is significantly affected by the proportion of various components therein. For this reason, the present inventors have conducted a lot of research, screening and optimization on the prescriptions of the injection emulsion containing N-suberoylanilide hydroxamic acid, and finally obtained an injection emulsion with excellent stability as described in the present invention, as well as a preparation method thereof.

Experiments have shown that the injection emulsion of the present invention can effectively increase the local effective concentration of N-suberoylanilide hydroxamic acid in lipophilic organs and/or tissues such as cardiovascular, and improve the bioavailability of N-suberoylanilide hydroxamic acid, which is advantageous for achieving an effective protection of the myocardium in the state of ischemia or reperfusion injury, and reducing or avoiding myocardial infarction during cardiac intervention operation. At the same time, since the local drug concentration of N-suberoylanilide hydroxamic acid in the heart and other tissues is successfully increased, general side effects are reduced or avoided, and the therapeutic effect of the drug on other diseases such as tumors is also improved.

### BRIEF DESCRIPTION OF THE DRAWINGS

Hereinafter, embodiments of the present invention will be described in detail with reference to the accompanying drawings, in which: Sham oral indicates a sham operated oral gavage group; Sham i.v indicates a sham operated intravenous injection group; I/R oral Pre indicates an ischemia/reperfusion one-time oral gavage group; I/R i.v Pre indicates an ischemia/reperfusion one-time intravenous injection group; I/R oral Pre+Reperfusion indicates an ischemia/reperfusion multiple-time oral gavage group; I/R i.v Pre+Reperfusion indicates an ischemia/reperfusion multiple-time intravenous injection group; MI oral indicates a myocardial infarction oral gavage group; and MI i.v indicates a myocardial infarction intravenous injection group.

FIG. 1 is a set of photographs showing the effect of N-suberoylanilide hydroxamic acid administered via oral gavage and intravenous injection on mice in the ischemia/reperfusion (I/R) group detected by echocardiography (24 h).

FIG. 2 shows the results of analysis of the effect of N-suberoylanilide hydroxamic acid administered via oral gavage and intravenous injection on mice in the ischemia/reperfusion (I/R) group detected by echocardiography (24 h); wherein * P<0.05, as compared with Sham oral; #P<0.05, as compared with I/R oral Pre; +P<0.05, as compared with I/R oral Pre+Reperfusion; n.s., no significant difference.

FIG. 3 is a set of photographs showing the effect of N-suberoylanilide hydroxamic acid administered via oral gavage and intravenous injection on mice in the myocardial infarction (MI) group detected by echocardiography (7 days, 14 days, and 28 days).

FIG. 4 shows the results of analysis of the effect of N-suberoylanilide hydroxamic acid administered via oral gavage and intravenous injection on mice in the myocardial infarction (MI) group detected by echocardiography (7 days, 14 days, and 28 days); wherein * P<0.05, as compared with Sham oral; #P<0.05, as compared with MI oral.

FIG. 5 is a set of photographs showing the effect of N-suberoylanilide hydroxamic acid administered via oral gavage and intravenous injection on mice in the ischemia/reperfusion (I/R) group detected by Evans blue/TTC double staining (24 h); wherein a non-ischemic area is shown in dark gray; an ischemic risk area is shown in light gray (the area circled by solid line); and a post-ischemia infarction area is shown in white (the area circled by broken line).

FIG. 6 shows the results of quantitative analysis of double staining area of cardiac tissue, illustrating the effect of N-suberoylanilide hydroxamic acid administered via oral gavage and intravenous injection on mice in the ischemia/reperfusion (I/R) group detected by Evans blue/TTC double staining (24 h); wherein * P<0.05, as compared with Sham oral; #P<0.05, as compared with I/R oral Pre; +P<0.05, as compared with I/R oral Pre+Reperfusion; n.s., no significance difference.

FIG. 7 is a set of photographs showing the effect of N-suberoylanilide hydroxamic acid administered via oral gavage and intravenous injection on mice in the myocardial infarction group detected by Masson staining (28 days); wherein normal myocardial tissue is shown in dark gray, and fibrous tissue is shown in light gray (the area circled by solid line).

FIG. 8 shows the results of analysis of the effect of N-suberoylanilide hydroxamic acid administered via oral gavage and intravenous injection on mice in the myocardial infarction group detected by Masson staining (28 days); wherein #P<0.05, as compared with MI oral.

FIG. 9 shows a photograph and the analysis results of the effect of N-suberoylanilide hydroxamic acid administered via oral gavage and intravenous injection on the acetylation levels of the cardiac tissue histones H3 and H4 of mice in the ischemia/reperfusion group detected by Western blot; wherein * P<0.05, as compared with Sham oral; #P<0.05, as compared with I/R oral Pre; +P<0.05, as compared with I/R oral Pre+Reperfusion; n.s., no significant difference.

FIG. 10 shows a photograph and the analysis results of the effect of N-suberoylanilide hydroxamic acid administered via oral gavage and intravenous injection on the acetylation levels of the cardiac tissue histones H3 and H4 of mice in the myocardial infarction group detected by Western blot; wherein * P<0.05, as compared with Sham oral; #P<0.05, as compared with MI oral.

FIG. 11 is a set of photographs showing the effects of injection emulsions with different prescriptions on mice in the ischemia/reperfusion (I/R) group detected by ultrasound (24 h).

FIG. 12 shows the results of analysis of the effects of injection emulsions with different prescriptions on mice in the ischemia/reperfusion (I/R) group detected by ultrasound (24 h); wherein * P<0.05, with prescriptions 1-6 compared with prescriptions 7-8.

FIG. 13 is a set of photographs showing the effects of injection emulsions with different prescriptions on mice in the ischemia/reperfusion (I/R) group detected by ultrasound (7 days, 14 days, and 28 days).

FIG. 14 shows the results of analysis of the effects of injection emulsions with different prescriptions on mice in the ischemia/reperfusion (I/R) group detected by ultrasound (7 days); wherein * P<0.05, with prescriptions 1-6 compared with prescriptions 7-8.

FIG. 15 shows the results of analysis of the effects of injection emulsions with different prescriptions on mice in the ischemia/reperfusion (I/R) group detected by ultrasound (14 days); wherein * P<0.05, with prescriptions 1-6 compared with prescriptions 7-8.

FIG. 16 shows the results of analysis of the effects of injection emulsions with different prescriptions on mice in the ischemia/reperfusion (I/R) group detected by ultrasound (28 days); wherein * P<0.05, with prescriptions 1-6 compared with prescriptions 7-8.

FIG. 17 is a set of photographs showing the effects of injection emulsions with different prescriptions on mice in the ischemia/reperfusion (I/R) group detected by Evans blue/TTC double staining (24 h); wherein a non-ischemic area is shown in dark gray; an ischemic risk area is shown in light gray (the area circled by solid line); and a post-ischemia infarction area is shown in white (the area circled by broken line).

FIG. 18 shows the results of quantitative analysis of double staining area of cardiac tissue, illustrating the effects of injection emulsions with different prescriptions on mice in the ischemia/reperfusion (I/R) group detected by Evans blue/TTC double staining (24 h); wherein * P<0.05, with prescriptions 1-6 compared with prescriptions 7-8.

FIG. 19 is a set of photographs showing the effects of injection emulsions with different prescriptions on mice in the myocardial infarction (MI) group detected by Masson staining (28 days); wherein a normal cardiac tissue area is shown in dark gray, and a fibrous tissue area formed after infarction is shown in light gray (the area circled by solid line).

FIG. 20 shows the results of analysis of the effects of injection emulsions with different prescriptions on mice in the myocardial infarction (MI) group detected by Masson staining (28 days); wherein * P<0.05, with prescriptions 1-6 compared with prescriptions 7-8.

FIG. 21 shows a photograph and the analysis results of the effects of injection emulsions with different prescriptions on the acetylation levels of the cardiac tissue histones H3 and H4 of mice in the ischemia/reperfusion (I/R) group detected by Western blot; wherein * P<0.05, with prescriptions 1-6 compared with prescriptions 7-8.

FIG. 22 shows a photograph and the analysis results of the effects of injection emulsions with different prescriptions on the acetylation levels of the cardiac tissue histones H3 and H4 of mice in the myocardial infarction (MI) group detected by Western blot; wherein * P<0.05, with prescriptions 1-6 compared with prescriptions 7-8.

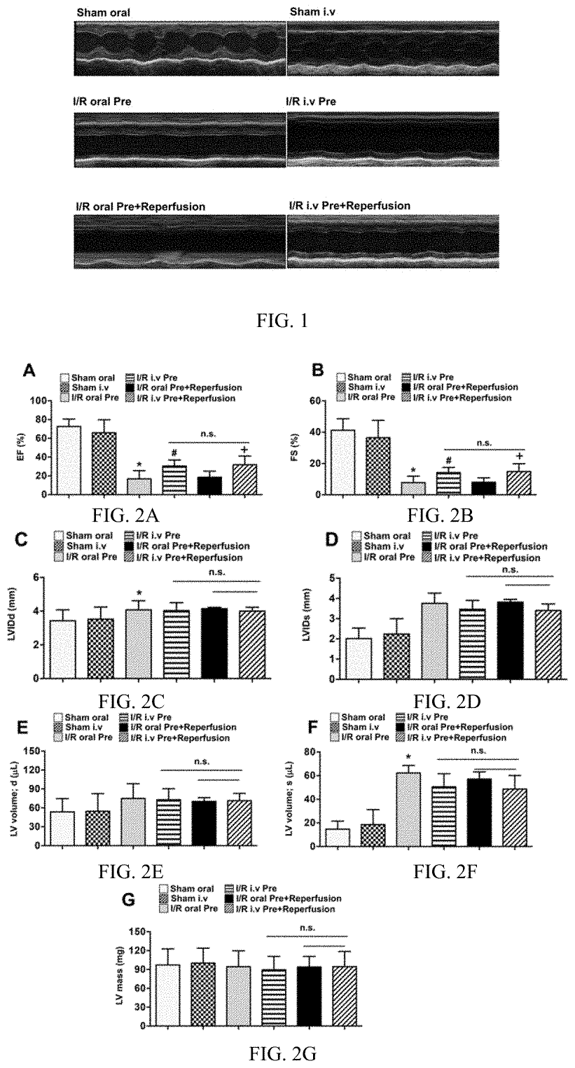

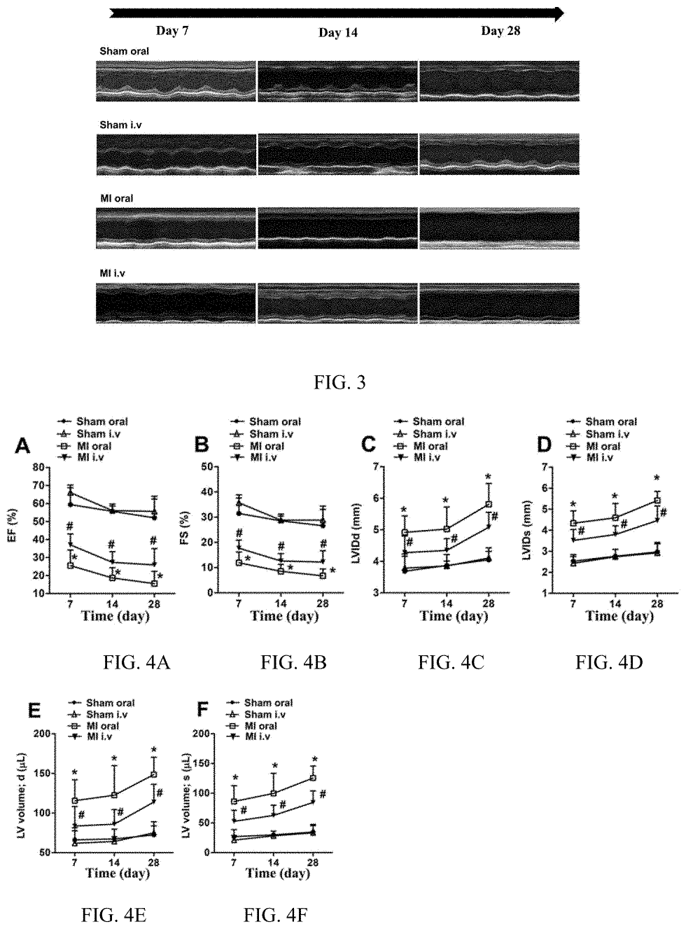

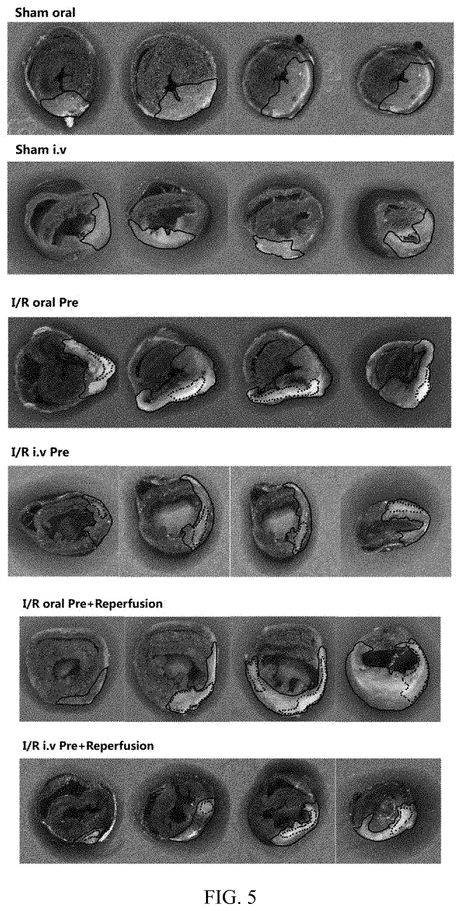

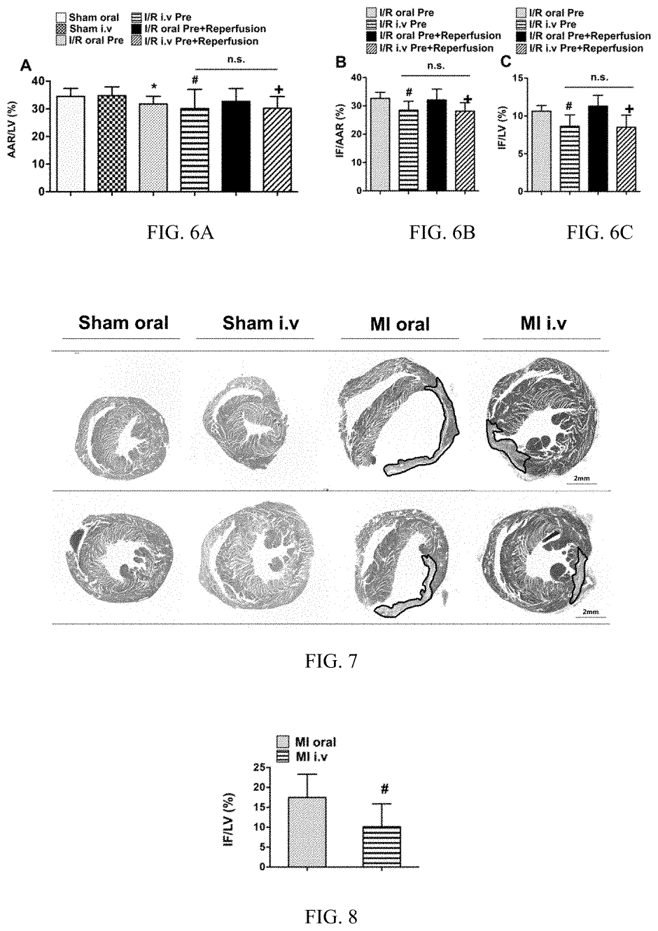

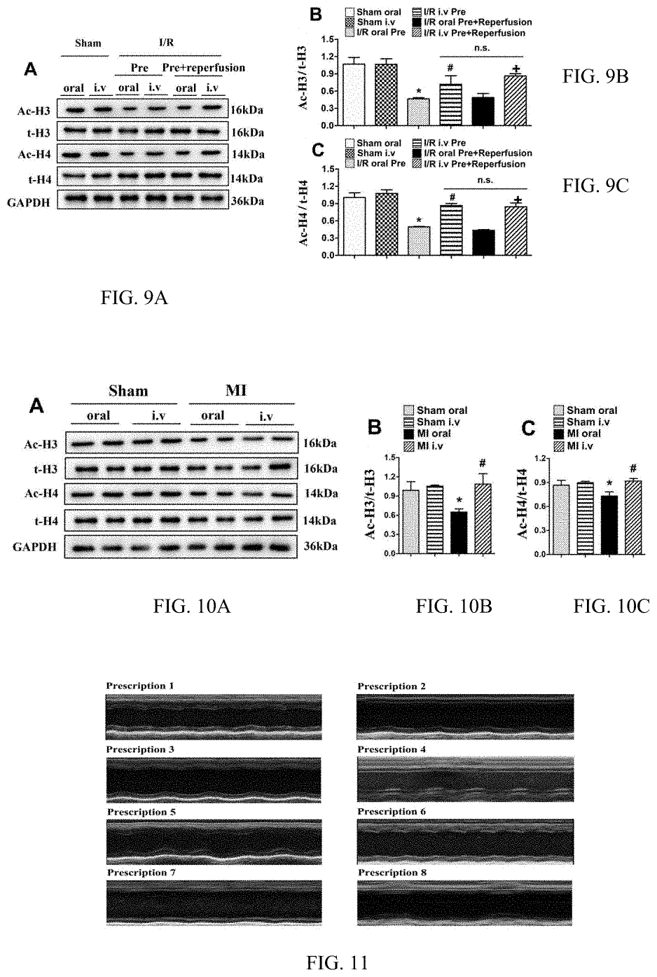

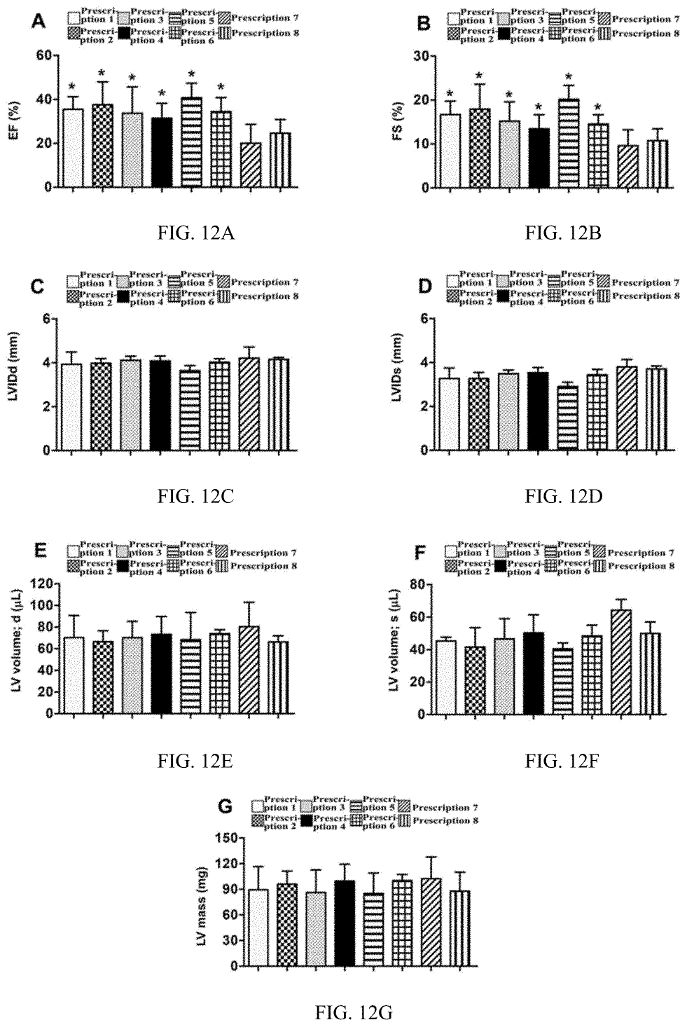

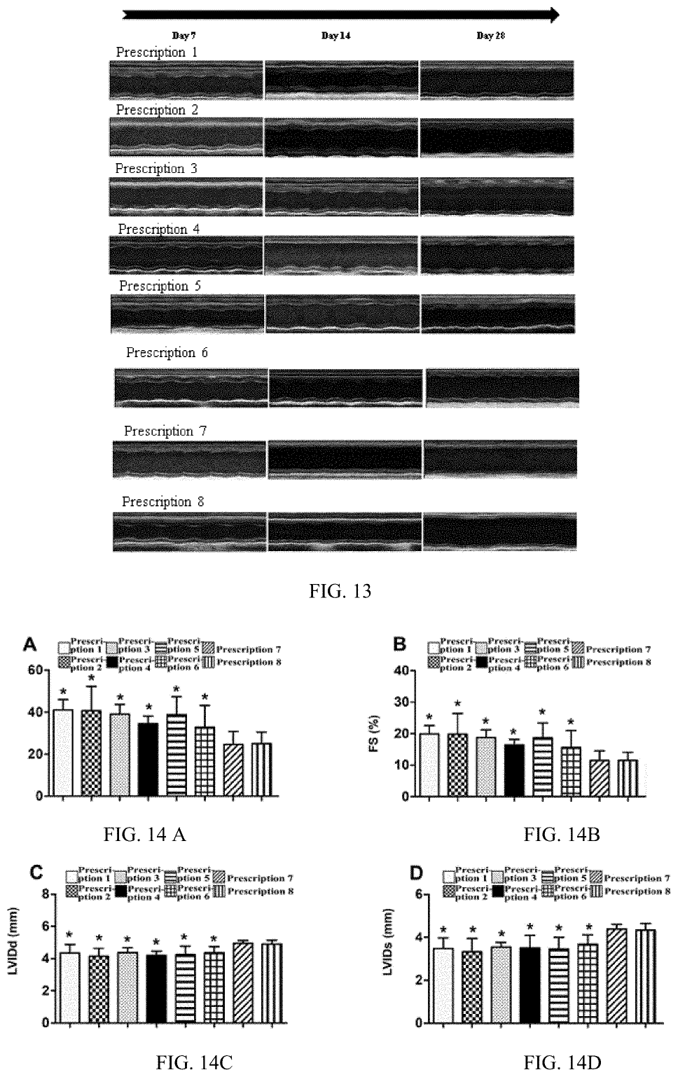

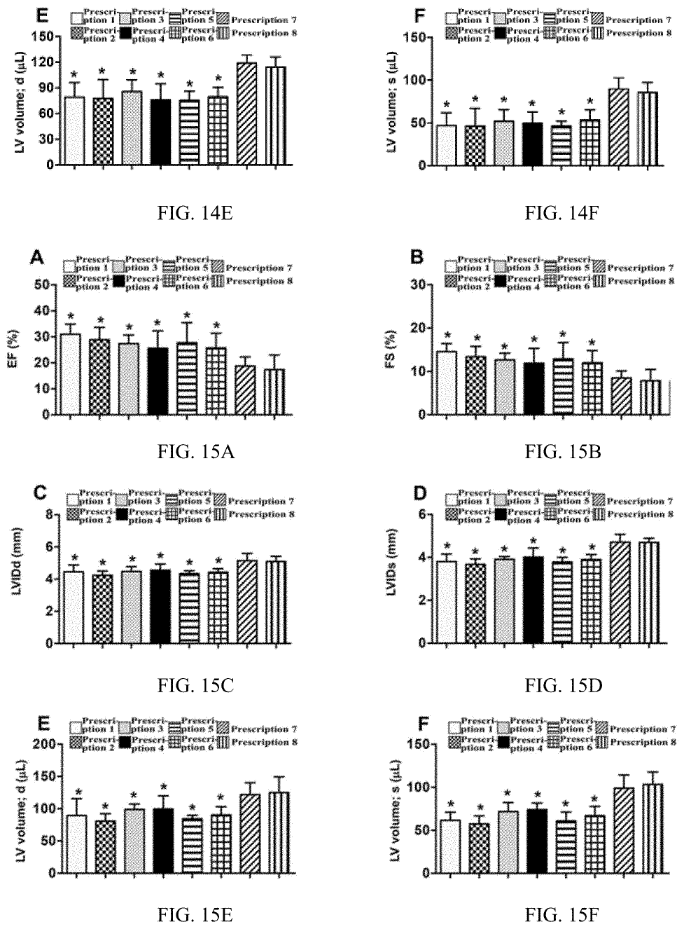

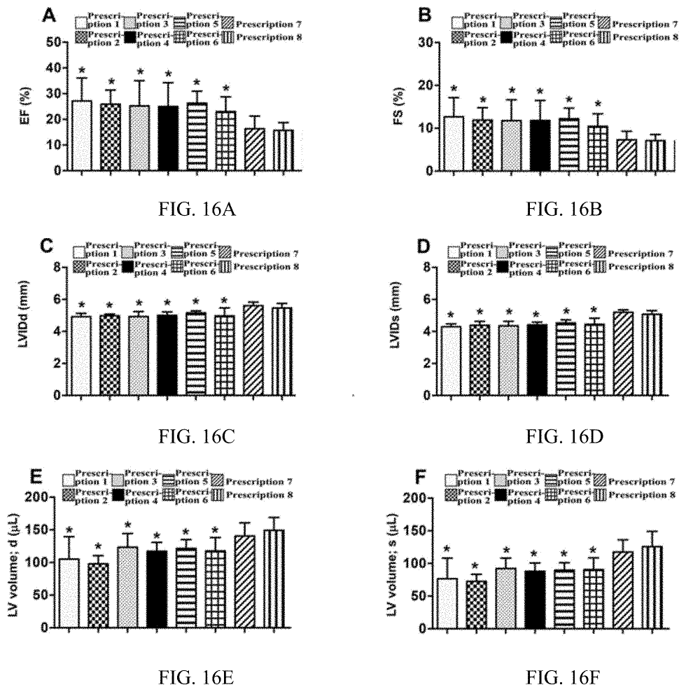

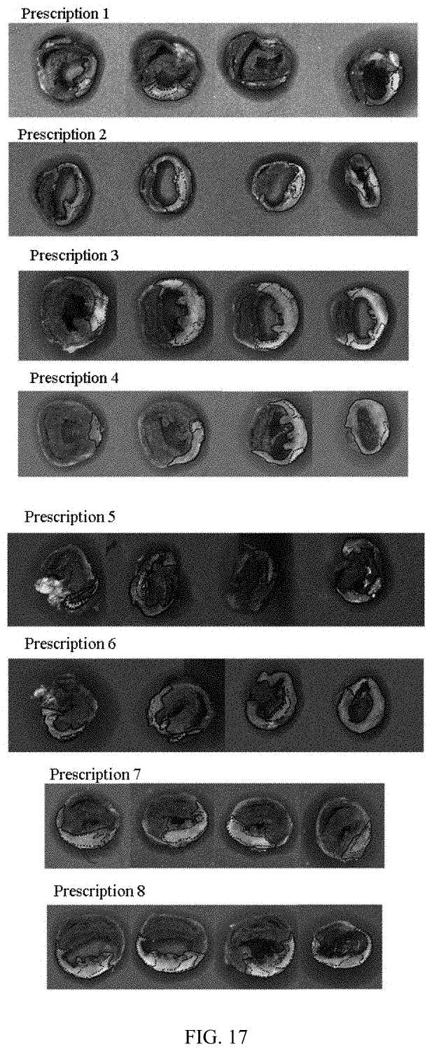

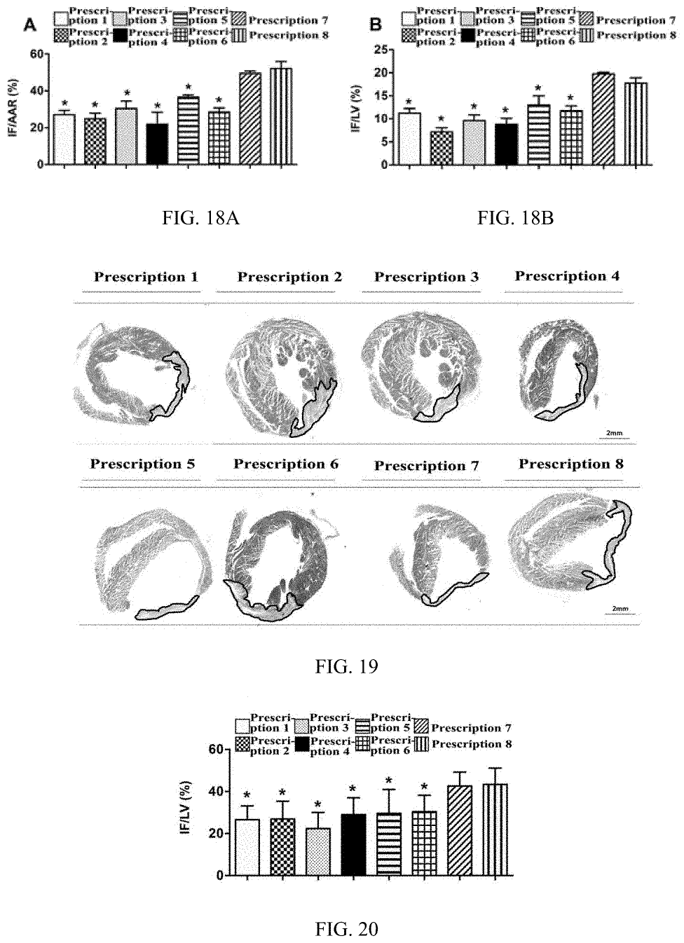

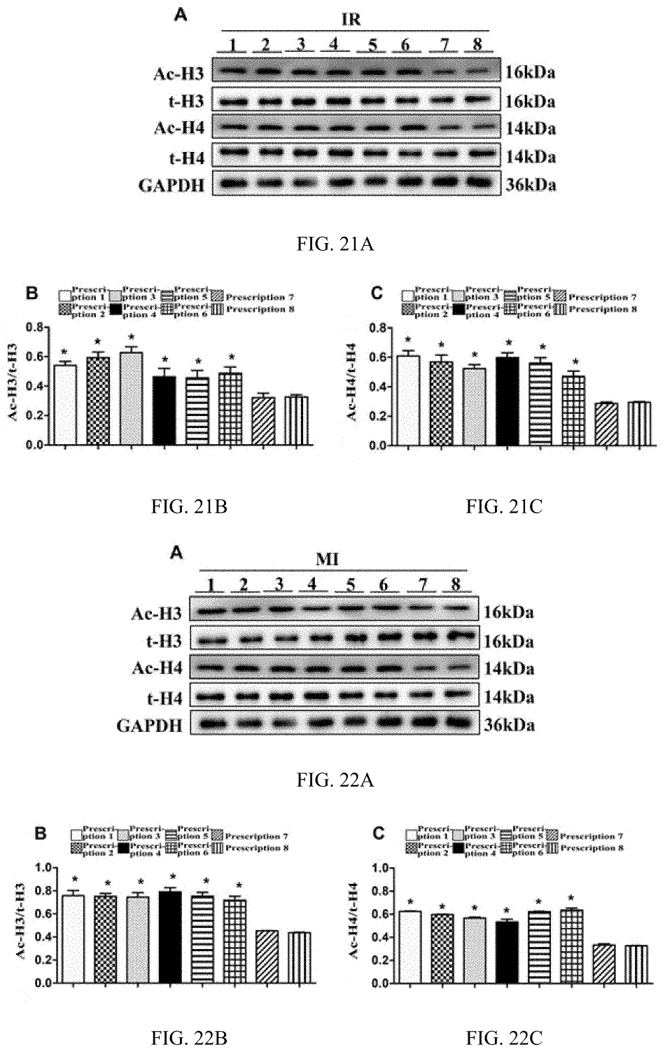

### DETAILED DESCRIPTION

The present invention will be described below with reference to specific examples. It can be understood by those skilled in the art that these examples are only intended to illustrate the present invention, and they do not limit the scope of the present invention in any way.

Unless otherwise specified, the experimental methods in the following examples are conventional methods. Unless otherwise specified, the raw materials and reagent materials used in the following examples are all commercially available products.

### Example 1

i) Prescription 1

| Name of raw and auxiliary materials | Prescribed amount (g) |
|---|---|
| N-suberoylanilide hydroxamic acid | 15 |
| soybean phospholipid | 10 |
| injection soybean oil | 100 |
| Tween-80 | 3 |
| oleic acid | 0.5 |
| glycerin | 12.5 |
| injection water | appropriate amount |

A volume of 500 ml is obtained.

ii) Process

A prescribed amount of N-suberoylanilide hydroxamic acid is weighed, Tween-80 and glycerin are added to form a mixture, which is then heated to 80° C., and about 100 ml of water is added to the mixture to dissolve the raw material drug; soybean phospholipid is added, shear-mixing (6000 rpm) is performed uniformly to obtain an aqueous phase; oleic acid and injection soybean oil are weighed and mixed uniformly to obtain an oil phase; the oil phase and the aqueous phase are shear-mixed at a high speed (6000 rpm) at 70° C. for 30 minutes to obtain a raw emulsion. The raw emulsion is homogenized under a pressure of 800 bar, cycled for 6 times, filled into an ampoule and sealed, and sterilized at 115° C. for 30 minutes to obtain the injection emulsion.

### Example 2

i) Prescription 2

| Names of raw and auxiliary materials | Prescribed amount (g) |
|---|---|
| N-suberoylanilide hydroxamic acid | 15 |
| soybean phospholipid | 5 |
| injection soybean oil | 75 |
| poloxamer 188 | 3 |
| oleic acid | 0.75 |
| glycerin | 12.5 |
| injection water | appropriate amount |

A volume of 500 ml is obtained.

ii) Process

A prescribed amount of N-suberoylanilide hydroxamic acid is weighed, poloxamer 188 and glycerin are added to form a mixture, which is then heated to 80° C., and about 100 ml of water is added to the mixture to dissolve the raw material drug; soybean phospholipid is added, shear-mixing (6000 rpm) is performed uniformly to obtain an aqueous phase; oleic acid and injection soybean oil are weighed and mixed uniformly to obtain an oil phase; the oil phase and the aqueous phase are shear-mixed at a high speed (5000 rpm) at 80° C. for 40 minutes to obtain a raw emulsion. The raw emulsion is homogenized under a pressure of 700 bar, cycled for 6 times, filled into an ampoule and sealed, and sterilized at 115° C. for 30 minutes to obtain the injection emulsion.

### Example 3

i) Prescription 3

| Names of raw and auxiliary materials | Prescribed amount (g) |
|---|---|
| N-suberoylanilide hydroxamic acid | 15 |
| soybean phospholipid | 12.5 |
| injection soybean oil | 50 |
| polyethylene glycol 15 | 5 |
| hydroxystearate | |
| oleic acid | 0.25 |
| glycerin | 11.5 |
| injection water | appropriate amount |

A volume of 500 ml is obtained.

ii) Process

A prescribed amount of N-suberoylanilide hydroxamic acid is weighed, polyethylene glycol 15 hydroxystearate and glycerin are added to form a mixture, which is then heated to 80° C., and about 100 ml of water is added to the mixture to dissolve the raw material drug; soybean phospholipid is added, shear-mixing (5000 rpm) is performed uniformly to obtain an aqueous phase; oleic acid and injection soybean oil are weighed and mixed uniformly to obtain an oil phase; the oil phase and the aqueous phase are shear-mixed at a high speed (6000 rpm) at 80° C. for 40 minutes to obtain a raw emulsion. The raw emulsion is homogenized under a pressure of 800 bar, cycled for 6 times, filled into an ampoule and sealed, and sterilized at 115° C. for 30 minutes to obtain the injection emulsion.

### Example 4

i) Prescription 4

| Names of raw and | Prescribed |
|---|---|
| auxiliary materials | amount (g) |
| N-suberoylanilide | 15 |
| hydroxamic acid | |
| soybean phospholipid | 11.5 |
| injection olive oil | 85 |
| Tween-80 | 3 |
| oleic acid | 0.6 |
| glycerin | 10 |
| injection water | appropriate amount |

A volume of 500 ml is obtained.

ii) Process

A prescribed amount of N-suberoylanilide hydroxamic acid is weighed, Tween-80 and glycerin are added to form a mixture, which is then heated to 70° C., and about 100 ml of water is added to the mixture to dissolve the raw material drug; soybean phospholipid is added, shear-mixing (6000 rpm) is performed uniformly to obtain an aqueous phase; oleic acid and injection olive oil are weighed and mixed uniformly to obtain an oil phase; the oil phase and the aqueous phase are shear-mixed at a high speed (6000 rpm) at 70° C. for 25 minutes to obtain a raw emulsion. The raw emulsion is homogenized under a pressure of 800 bar, cycled for 6 times, filled into an ampoule and sealed, and sterilized at 115° C. for 30 minutes to obtain the injection emulsion.

### Example 5

i) Prescription 5

| Names of raw and auxiliary materials | Prescribed amount (g) |
|---|---|
| N-suberoylanilide hydroxamic acid | 15 |
| lecithin | 10 |
| injection medium-chain oil | 100 |
| Tween-80 | 0.5 |
| oleic acid | 0.5 |
| glycerin | 12.5 |
| injection water | appropriate amount |

A volume of 500 ml is obtained.

ii) Process

A prescribed amount of N-suberoylanilide hydroxamic acid is weighed, Tween-80 and glycerin are added to form a mixture, which is then heated to 80° C., and about 100 ml of water is added to the mixture to dissolve the raw material drug; lecithin is added, shear-mixing (5000 rpm) is performed uniformly to obtain an aqueous phase; oleic acid and injection medium-chain oil are weighed and mixed uniformly to obtain an oil phase; the oil phase and the aqueous phase are shear-mixed at a high speed (6000 rpm) at 70° C. for 30 minutes to obtain a raw emulsion. The raw emulsion is homogenized under a pressure of 900 bar, cycled for 5 times, filled into an ampoule and sealed, and sterilized at 115° C. for 30 minutes to obtain the injection emulsion.

### Example 6

i) Prescription 6

| Names of raw and auxiliary materials | Prescribed amount (g) |
|---|---|
| N-suberoylanilide hydroxamic acid | 15 |
| soybean phospholipid | 10 |
| injection soybean oil | 100 |
| propylene glycol | 3 |
| oleic acid | 0.5 |
| glycerin | 12.5 |
| injection water | appropriate amount |

A volume of 500 ml is obtained.

ii) Process

A prescribed amount of N-suberoylanilide hydroxamic acid is weighed, glycerin and propylene glycol are added to form a mixture, which is then heated to 70° C., and about 100 ml of water is added to the mixture to dissolve the raw material drug; soybean phospholipid is added, shear-mixing (6000 rpm) is performed uniformly to obtain an aqueous phase; oleic acid and injection soybean oil are weighed and mixed uniformly to obtain an oil phase; the oil phase and the aqueous phase are shear-mixed at a high speed (5000 rpm) at 70° C. for 30 minutes to obtain a raw emulsion. The raw emulsion is homogenized under a pressure of 800 bar, cycled for 6 times, filled into an ampoule and sealed, and sterilized at 115° C. for 30 minutes to obtain the injection emulsion.

### Comparative Example 1

i) Prescription 7

| Names of raw and auxiliary materials | Prescribed amount (g) |
|---|---|
| N-suberoylanilide hydroxamic acid | 15 |
| soybean phospholipid | 10 |
| injection soybean oil | 100 |
| Tween-80 | 3 |
| vitamin E | 0.5 |
| glycerin | 12.5 |
| injection water | appropriate amount |

A volume of 500 ml is obtained.

ii) Process

A prescribed amount of N-suberoylanilide hydroxamic acid is weighed, glycerin and Tween-80 are added to form a mixture, which is then heated to 80° C., and about 100 ml of water is added to the mixture to dissolve the raw material drug; soybean phospholipid is added, shear-mixing (6000 rpm) is performed uniformly to obtain an aqueous phase; vitamin E and injection soybean oil are weighed and mixed uniformly to obtain an oil phase; the oil phase and the aqueous phase are shear-mixed at a high speed (6000 rpm) at 70° C. for 30 minutes to obtain a raw emulsion. The raw emulsion is homogenized under a pressure of 800 bar, cycled for 6 times, filled into an ampoule and sealed, and sterilized at 115° C. for 30 minutes to obtain the injection emulsion.

### Comparative Example 2

i) Prescription 8

| Names of raw and auxiliary materials | Prescribed amount (g) |
|---|---|
| N-suberoylanilide hydroxamic acid | 15 |
| soybean phospholipid | 10 |
| injection soybean oil | 100 |
| Tween-80 | 3 |
| polyoxyethylene hydrogenated | 0.5 |
| castor oil | |
| glycerin | 12.5 |
| injection water | appropriate amount |

A volume of 500 ml is obtained.

ii) Process

A prescribed amount of N-suberoylanilide hydroxamic acid is weighed, Tween-80 and glycerin are added to form a mixture, which is then heated to 80° C., and about 100 ml of water is added to the mixture to dissolve the raw material drug; soybean phospholipid is added, shear-mixing (6000 rpm) is performed uniformly to obtain an aqueous phase; polyoxyethylene hydrogenated castor oil and injection soybean oil are weighed and mixed uniformly to obtain an oil phase; the oil phase and the aqueous phase are shear-mixed at a high speed (6000 rpm) at 70° C. for 30 minutes to obtain a raw emulsion. The raw emulsion is homogenized under a pressure of 800 bar, cycled for 6 times, filled into an ampoule and sealed, and sterilized at 115° C. for 30 minutes to obtain the injection emulsion.

Examples of Pharmacological Experiments

Establishment of Animal Models

1.1 Experimental Materials:

one pair of ophthalmic straight scissors, one pair of ophthalmic curved scissors, one pair of toothed curved forceps, one pair of toothed straight forceps, one pair of non-toothed curved forceps, one pair of non-toothed straight forceps, one mosquito vascular clamp, several suture threads with needles (6/0), several emesis basins, several gauzes, several sterile cotton swabs, physiological saline, an inhalation gas anesthesia machine, 10 ml syringes, 50 ml syringe, mouse gavage needles, a mouse operating table, a thermal pad, 1.5 ml centrifuge tubes, 15 ml cryotubes, cell culture dishes, surgical blades, scalpel handles, 2% isoflurane.

1.2 Establishment of Sham and Ischemia/Reperfusion (I/R) Mouse Model

After the mice were anesthetized by inhaling 2% isoflurane, a skin incision of about 1.2 cm is made in the left anterior chest wall of the mice and a purse-string suture is reserved. Chest muscle tissue is bluntly separated in the purse, which exposes the fourth intercostal space of the left chest. Next, the pleural cavity and the pericardial cavity are gently and rapidly opened in the purse using the mosquito vascular clamp at the position of the fourth intercostal space to squeeze out the heart. A slipknot is ligated at the anterior descending branch of the left coronary artery of the heart by suture threads with needles (6/0). When it is observed through naked eyes that the color of myocardial tissue in the corresponding area turns to be gray, it can be confirmed that the ligation is successful. After that, the heart is quickly put back into the chest cavity, the air is completely drained from the cavity, the purse is tightened to close the chest cavity, muscle and skin, and a thread end of the slipknot for ligating the left anterior descending branch is left outside the chest. The mice are placed in a ventilated environment to monitor their recovery conditions. After 45 minutes of ischemia, the myocardial tissue is reperfused with blood by softly and gently dragging the thread end left outside the chest to loosen the slipknot. For the control sham-operated mice, operations are the same as those on the ischemia/reperfusion mice except that the anterior descending branch is not ligated by a slipknot for the control sham mice.

1.3 Establishment of Sham and Myocardial Infarction (MI) Mouse Model

After the mice were anesthetized by inhaling 2% isoflurane, a skin incision of about 1.2 cm is made in the left anterior chest wall of the mice and a purse-string suture is reserved. Chest muscle tissue is bluntly separated in the purse, which exposes the fourth intercostal space of the left chest. Next, the pleural cavity and the pericardial cavity are gently and rapidly opened in the purse using the mosquito vascular clamp at the position of the fourth intercostal space to squeeze out the heart. A slipknot is ligated at ligated at the anterior descending branch of the left coronary artery of the heart by suture threads with needles (6/0). When it is observed through naked eyes that the color of myocardial tissue in the corresponding area turns to be gray, it can be confirmed that the ligation is successful. After that, the heart is quickly put back into the chest cavity, the air is completely drained from the cavity, and the purse is tightened to close the chest cavity, muscle and skin. For the control sham-operated mice, operations are the same as those on the myocardial infarction mice except that the anterior descending branch is not ligated for the control sham mice.

Detection Method

2.1 Ultrasound Detection of Cardiac Function

Mice in the ischemia/reperfusion group: the mice are anesthetized by inhaling 2% isoflurane again 24 hours after the operation.

1-15. (canceled)

16. An injection emulsion for protecting ischemic myocardium, comprising N-suberoylanilide hydroxamic acid, emulsifier, injection oil, solubilizer, oleic acid, glycerol and injection water.

17. The injection emulsion according to claim 16, comprising, in parts by weight:
1˜5 parts of N-suberoylanilide hydroxamic acid;
0.2˜12.5 parts of emulsifier;
2˜100 parts of injection oil;
0.02˜5 parts of solubilizer;
0.03˜0.4 parts of oleic acid;
0.4˜12.5 parts of glycerol; and
balance: injection water.

18. A method for prevention and/or treatment of a heart disease wherein said method comprises administering, to a subject in need of such prevention and/or treatment, a medicament comprising an N-suberoylanilide hydroxamic acid emulsion.

19. The method according to claim 18, wherein the emulsion comprises N-suberoylanilide hydroxamic acid, emulsifier, injection oil, solubilizer, oleic acid, glycerol and injection water.

20. The method according to claim 18, wherein medicament comprises, in parts by weight:
1˜5 parts of N-suberoylanilide hydroxamic acid;
0.2˜12.5 parts of emulsifier;
2˜100 parts of injection oil;
0.02˜5 parts of solubilizer;
0.03˜0.4 parts of oleic acid;
0.4˜12.5 parts of glycerol; and
balance: injection water.

21. The method according to claim 18, wherein the heart disease is ischemia-reperfusion injury.

22. The method according to claim 18, wherein the heart disease is myocardial ischemia.

23. The method according to claim 18, wherein the heart disease is myocardial infarction.

24. The method according to claim 18, wherein the medicament if formulated as an injection emulsion.

25. The method according to claim 18, wherein the medicament is administered by injection.

26. A method for preparing an injection emulsion, wherein the method comprises the following steps:
(1) adding a solubilizer and glycerol to N-suberoylanilide hydroxamic acid, heating, and then adding injection water to dissolve N-suberoylanilide hydroxamic acid;
(2) adding an emulsifier to the solution obtained in step (1) and uniformly mixing to obtain an aqueous phase;
(3) uniformly mixing oleic acid with an injection oil to obtain an oil phase;
(4) shear-mixing the aqueous phase and the oil phase at a high speed while heating to obtain a raw emulsion; and
(5) homogenizing the raw emulsion under pressure to obtain the injection emulsion.
